(19) United States
(12) Patent Application Publication
(10) Pub. No.: US 2012/0222172 A1
(43) Pub. Date: Aug. 30, 2012

(54) YELLOW DWARF VIRUS AND FUSARIUM HEAD BLIGHT RESISTANCE INTROGRESSED AND COMBINED IN WHEAT

(75) Inventor: Herbert W. Ohm, West Lafayette, IN (US)

(73) Assignee: PURDUE RESEARCH FOUNDATION, West Lafayette, IN (US)

(21) Appl. No.: 13/508,252

(22) PCT Filed: Nov. 23, 2010
(86) PCT No.: PCT/US2010/057845

§ 371 Date: May 4, 2012

Related U.S. Application Data
(60) Provisional application No. 61/263,473, filed on Nov. 23, 2009.

(51) Int. Cl. A01H 5/10; A01H 5/00

(52) U.S. Cl. 800/301; 800/320.3

(57) ABSTRACT

Wheat plants, seeds and plant parts are disclosed that are resistant to both yellow dwarf virus (YD) and fusarium head blight (FHB).

### CROSS REFERENCE TO RELATED APPLICATIONS

This application claims priority to U.S. Provisional Patent Application No. 61/263473, filed Nov. 23, 2009, the content of which application is incorporated herein by reference in its entirety.

The United States Government has rights in this invention pursuant to USDA/ARS Grant Number 58-3620-8-388.

SEQUENCE LISTING

The instant application contains a Sequence Listing which has been submitted in ASCII format via EFS-Web and is hereby incorporated by reference in its entirety. Said ASCII copy, created on Nov. 23, 2010, is named 113700_SEQ.txt and is 2,779 bytes in size.

### BACKGROUND AND SUMMARY OF THE DISCLOSURE

Wheat is disclosed that is resistant to both yellow dwarf virus (YD) and fusarium head blight (FHB).

Yellow dwarf (YD) disease, caused by luteoviruses that are transmitted by aphids, Rhopalosiphum padi; and fusarium head blight (FHB) disease, caused by the fungus Fusarium graminearum, are globally important diseases of wheat. The highly effective resistance gene (Bdv3) to YD and the highly effective resistance QTL (Qfhs.pur-7EL) to FHB, were introgressed into wheat from related grass species and then combined in wheat by classical plant breeding—mating of parent plants and genetic recombination. Bdv3 was introgressed into wheat chromosome 7D from intermediate wheatgrass (Thinopyrum intermedium) chromosome 7E, in the Purdue University wheat germplasm line P961341 (USDA-ARS GP-780, PI 634825) and commercialized in the Purdue University wheat cultivars INW0316 and INW0801. Qfhs.pur-7EL was introgressed into wheat chromosome 7D from tall wheatgrass (Th. ponticum) chromosome 7E in the Purdue University wheat line 275-4. The introgressed chromosome 7E segments with, respectively, Bdv3 and Qfhs.pur-7EL, replace the distal approximately ⅓ of the long arm of 7D, but in different wheat genotypes. Thus, the two resistance factors are in repulsion—only one or the other factors are present in any wheat line that existed prior to that disclosed herein.

Those of skill in the art, that is competent wheat breeder/geneticists can combine Bdv3 and Qfhs.pur-7EL. For example, they would obtain the wheat line P961341, which is registered in the USDA-ARS wheat germplasm repository, or one of the wheat cultivars that Purdue has released commercially, e.g., INW0316—that have Bdv3. They would also obtain seeds of the wheat lines 275-4 or a wheat line derived from 275-4. Crosses to combine the two traits are made and selection for the respective traits phenotypically and genotypically use the respective co-segregating markers described herein.

A goal was combining yellow dwarf virus, and fusarium head blight, UG99 stem and rust and stripe resistance genes in soft winter wheat adapted to the eastern USA. Two genetic resistance factors, Bdv3 and Qfhs.pur-7EL were combined in coupling, so that both factors were present in a given wheat line. Bdv3 is located more proximal to the centromere of chromosome 7D than Qfhs.pur-7EL, which made it possible to generate a meiotic chromosomal recombination in the distal region of chromosome 7DS.7DL.7EL to produce a progeny plant in which Bdv3 and Qfhs.pur-7EL were combined in the same chromosome. Consequently, both resistance factors were combined (in coupling) in the same lineage. Because there is very infrequent recombination between the homoeologous (nonhomologous) chromosomes 7D and 7E, the two resistance factors will remain in coupling in wheat.

BDV3 is the marker that co-segregates with gene Bdv3. Since this marker is located on the introgressed chromosome 7E segment that carries Bdv3, the marker will remain associated with Bdv3 in any wheat background, because there is no or little recombination between 7E and 7D—the wheat chromosome into which the 7E segment was introgressed.

The markers BE445653 and BF145935 are on the proximal (toward the centromere of chromosome 7D) side of Qfhs.pur-7EL that is on the 7E segment that was introgressed into the distal end of chromosome 7DL. The marker cfa2240 is at the distal end of Qfhs.pur-7EL. These three markers will remain associated with Qfhs.pur-7EL in any wheat background, because, like the segment that carries Bdv3, there is no or little recombination between 7E and 7D in wheat. Thus, when cfa2240 and either BE445653 or BF145935 (markers that flank the Qfhs.pur-7EL) are present that Qfhs.pur-7EL is certainly present. (See the chromosome map, FIG. 4).

### BRIEF DESCRIPTION OF THE DRAWINGS

FIG. 1 shows wheat plants resistant to yellow dwarf disease (YD) in naturally infected field plots a) Resistant line with the YD-resistance gene, Bdv3; which showed green leaves and a healthy appearance; and b) Susceptible line without Bdv3, or any other YD-resistance gene(s), which showed brown leaves and a dead appearance. (Photo: Purdue University Agronomy Center for Research and Education.)

FIG. 2. is a diagram of wheat chromosome 7D with introgressed segments carrying Bdv3 and Qfhs.pur-7EL. Bdv3 was introgressed into wheat chromosome 7DL from intermediate wheatgrass (Thinopyrum intermedium) and is located subterminal on the 7E segment that was translocated into 7DS.7DL-7EL. Qfhs.pur-7EL was introgressed from tall wheatgrass (Thinopyrum ponticum) and was mapped to the distal region of chromosome 7DS.7DL-7EL in wheat (Shen and Ohm, 2007, p. 132, 134-135). Bdv3 is located more proximal to the centromere of chromosome 7DS.7DL-7EL than Qfhs.pur-7EL.

FIG. 3. Resistant wheat head, with Qfhs.pur-7EL, infected with fusarium head blight (FHB). Note that the infection did not spread past the infected glume (circled). Purdue University Agronomy Center for Research and Education.

FIG. 4 shows 3 resistant stalks and one susceptible stalk. The resistant lines are green and full. The susceptible line is brownish. The 3 FHB resistant spikes have Qfhs.pur-7EL and gene Fhb1 (their resistance is augmented when the two resistance factors are combined, both are present in the plant). The flower near the tip of the spikes on each plant that is marked with a black pen (circled) was inoculated with Fusarium graminearum spores at flowering: the disease did not spread beyond the inoculated flower in the 3 resistant spikes, but the whole spike of the susceptible plant became diseased.

FIG. 5 Comparative maps of the long arm of homoeologous group 7: The genetic map of 7E disclosed herein and the deletion bin map of wheat 7A, 7B, and 7D. Location of markers in deletion bin is according to Hossain et al (2004) and Sourdille et al. (2004). Marker order within the same bin is not known.

FIG. 6 Schematic diagram showing the presence (grey area) or absence (white area) of 7E chromosomal segments in either BYDV susceptible or resistant translocation lines (TLs). The 7E chromosomal segments in the TLs were arranged assuming the colinearity of Triticeae group 7 markers to the 7E chromosome present in P29; the progenitor of these TLs (Francki et al. 1997). However, it is not known at this point if the wheatgrass translocations are attached to wheat chromosomes other than 7D. The map location of the RFLP markers Used in this study as shown with linkage distances (cM) were taken from either the wheat 7D (Gale et al., 1995) or Triticeae group 7 (T7) (Van Deynze et at., (1995) maps. The estimated breakpoints for the segments were placed at half the distance to the neighboring markers. The asterisks indicate that data for that marker for that line was not determined. The location of the BYDV resistance locus (BYDVR) was derived from comparing the presence or absence of 7E chromosomal segments between resistant and susceptible TLs and 254-2, a susceptible (S) translocation line that did not contain the wheatgrass-specific telomere repetitive sequence. [Crasta et al. (2000) Genome 43; 698-706].

### DETAILED DESCRIPTION OF THE DISCLOSURE

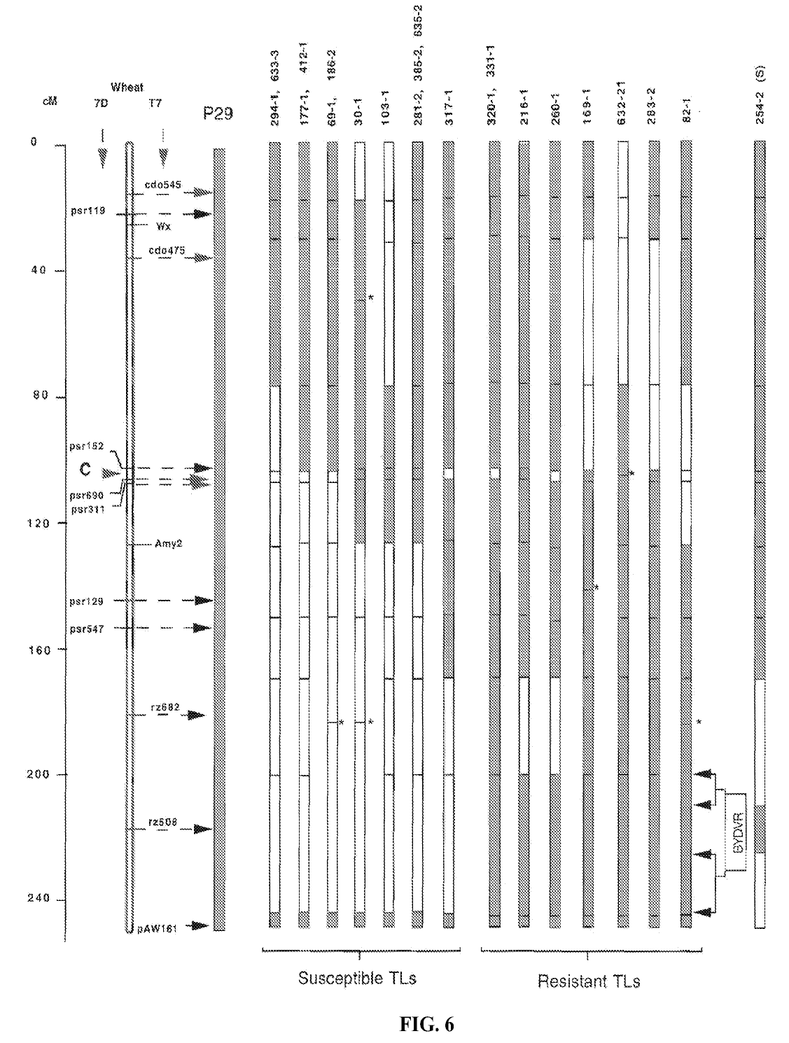

As part of a wheat improvement research program, a highly effective resistance to yellow dwarf disease (YD), gene Bdv3 was identified in intermediate wheatgrass (YD) and a highly effective gene/QTL, Qfhs.pur-7EL, for type 2 resistance to fusarium head blight (FHB) was identified in tall wheatgrass. The Bdv3 gene has not been sequenced, because there is no recombination between the introgressed chromosome segment carrying Bdv3 and corresponding wheat DNA. These genes were both introgressed into wheat on chromosome 7DL (Sharma et al., 1995, p. 424-425, 428-429; Shen and Ohm, 2006). Qfhs.pur-7EL augments FHB resistance when combined with FHB resistance genes that are native to common wheat (Shen and Ohm, 2006). Introgressed 7EL segment containing Bdv3 and the introgressed 7E segment containing Qfhs.pur-7EL both replaced the distal approximately ⅓ of 7DL. Bdv3 is located more proximal to the centromere on 7DL than is Qfhs.pur-7EL (Crasta, et al., 2000). The Crasta article documented that Bdv3 is subterminal (FIG. 6) and the Shen and Ohm article showed that Qfhs.pur-7EL is very near the terminal end of the chromosome. (Ohm et al. 2005; Shen and Ohm, 2007). A genetic recombination in the 7E region between the two loci, Bdv3 and Qfhs.pur-7EL was effected.

### Localization of BYDV resistance genes on chromosome 7E

The 7E chromosomal segments of all RDLs and SOLs were arranged according to their relative positions on the wheat group 7 map (FIG. 6). Comparison of the seven resistant and 12 susceptible translocation lines containing varying and overlapping amounts of alien chromatin identified a small chromosomal segment conferring BYOV resistance near the distal end of chromosome 7E. The 7E chromosomal segment containing the wheat group 7L marker, rz508, was present in all the RDLs but was absent in all the SOLs containing the 7EL telomere-specific repetitive sequence (FIG. 6). These results provided strong evidence that the resistance gene(s) were located in the vicinity of this marker on the distal end of chromosome 7EL. The independently identified SDL, 254-2, which did not contain the 7EL telomere-specific repetitive sequence (data not shown), contained the rz508 marker but not its neighboring marker rz682 (FIG. 6). Ordering of the 7E chromosomal segments in all RDLs and SOLs according to their relative positions in wheat group 7 chromosomes localized the BYDV resistance genes to the interstitial region between rz682 and the telomere (FIG. 6). [Crasta et al. (2000) Genome 43; 698-706].

### New Wheat Lines

The wheat line, 275-4, was developed in which the introgressed 7E segment from Thinopyrum ponticum containing Qfhs.pur-7EL replaced the distal ⅓ of the wheat chromosome 7DL in a Chinese spring (CS) wheat background, determined by absence of SSR markers mapped to 7DL and presence of markers diagnostic for 7EL. A wheat line containing the introgressed segment from P961341 and the line 275-4, with Qfhs.pur-7EL, were crossed.

Seeds of wheat line P961341 containing Bdv3, and seeds of wheat line P275-4 containing Qfhs.pur-7EL, were planted in flats and vernalized for 65 days, then transplanted to a greenhouse, and the two parent lines were mated to produce F1 seeds that were heterozygous for chromosome 7DS.7DL-7EL containing Bdv3 and chromosome 7DS.7DL-7EL containing Qfhs.pur-7EL.

A wheat line 07117B1-29-7-9-9-4-5 was developed by crossing a line that has Qfhs.pur-7EL combined with Bdv3 X a line that has Fhb1 and then backcrossed the F1 plants X the line that has Fhb1, and using respective co-segregating markers in subsequent generations of inbreeding, a true-breeding (homozygous) line (07117B1-29-7-9-9-4-5) was identified that has Qfhs.pur-7EL, Bdv3 and Fhb1.

### Plant Genotyping

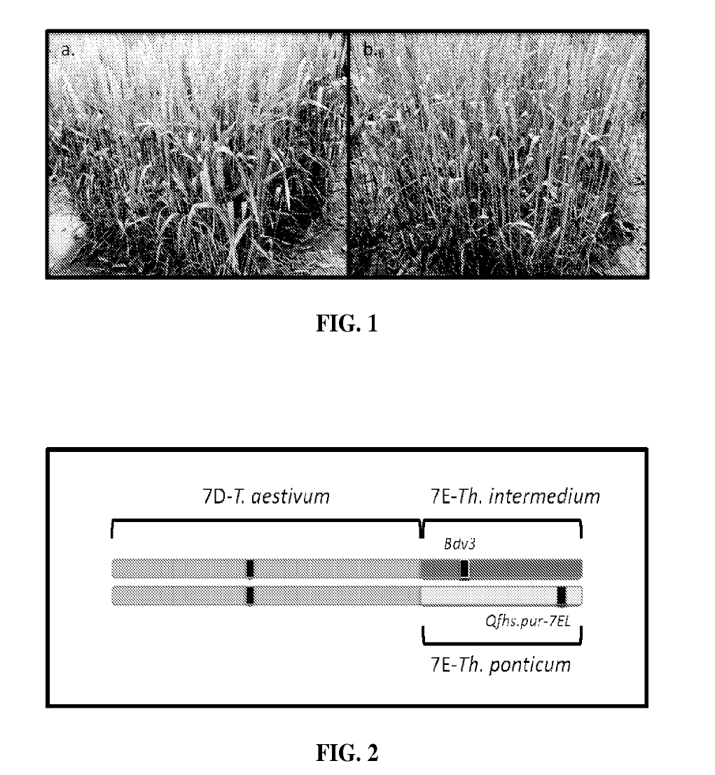

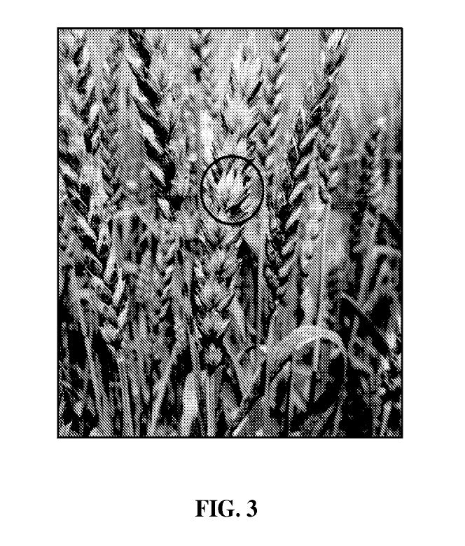

The F₁ plants were grown in a greenhouse for 4 months producing F₂ seeds (segregating population). The resulting F₂ plants were vernalized in a refrigerated chamber with fluorescent lights with a 12-hour day/night cycle for, the next 2 months, then transplanted to a field in the following month. Plants that were heterozygous for Bdv3 and Qfhs.pur-7EL (FIG. 2) were identified based on co-segregating and co-dominant simple sequence repeat (SSR) markers: gwm37 (Ayala et al., 2001) for Bdv3 and the flanking markers cfa2240 and BF145935 (Shen and Ohm, 2007, p. 133-135, Table 1) for Qfhs.pur-7EL.

Leaf tissue samples were collected prior to flowering, and the samples were genotyped by the USDA-ARS Regional Genotyping facility in Raleigh, N.C. using high-throughput marker screening to identify plants that were positive for Bdv3 using the marker BDV3 (Kong et al., 2009). The FHB-resistance QTL, Qfhs.pur-7EL, was detected using the distal EST-derived marker BF145935 and the proximal SSR marker cfa2240. Fhb1, a FHB-resistance gene located on chromosome 3B (Anderson et al., 2001, pp. 1165-1168) was detected using the flanking SSR markers barc008 (Song et al., 2002, Table 4) and gwm533 (Roder et al., 1998, Gatersleben wheat-microsatellite, FIG. 1). F_{2:3} seed from the selected plants was planted in the field in 1-m² plots.

### Plant Phenotyping

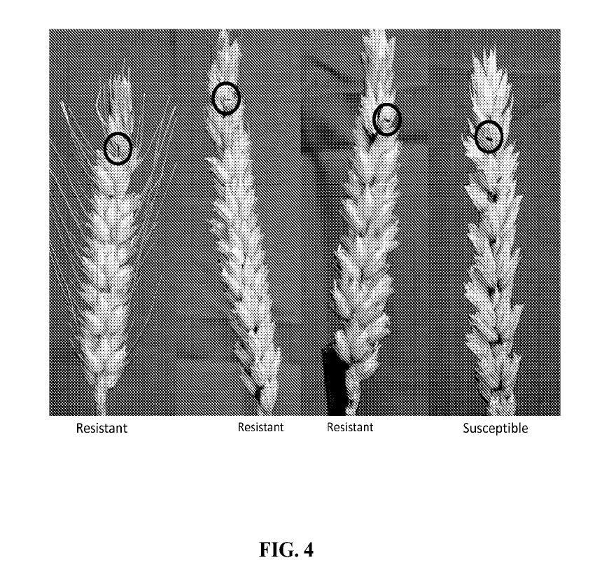

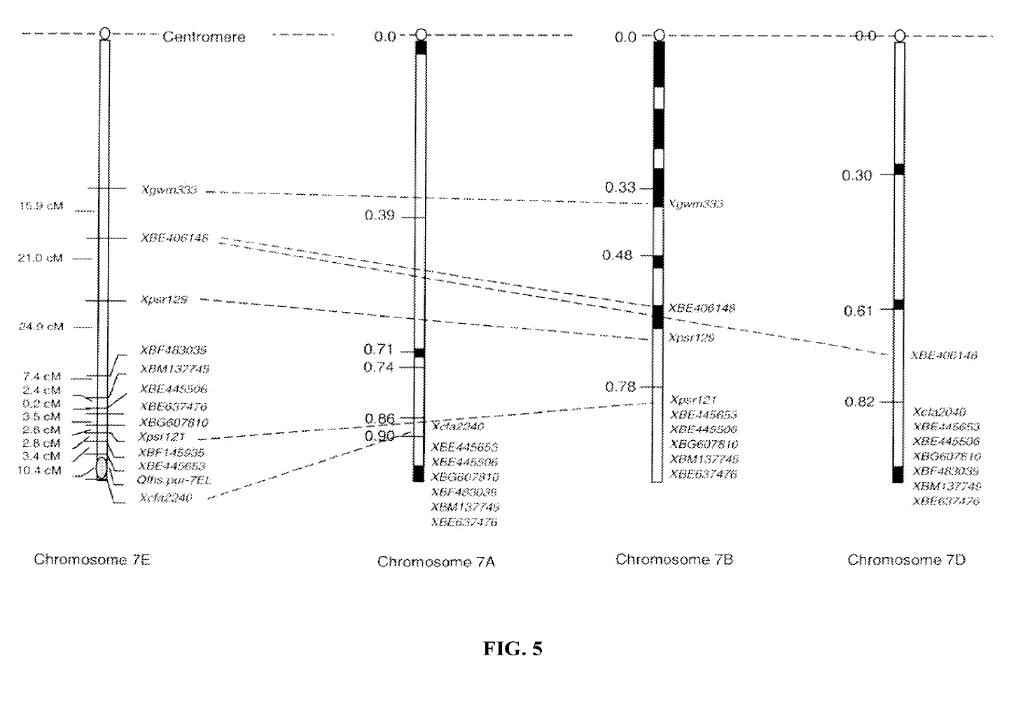

The next month, ten F_{2:3} seedlings from each of 34 1-m² plots in a field nursery were dug, transferred to small pots and vernalized in a cold room at 2° C., 12-hour light, for 65 days. The plants were then transplanted individually to 10-cm pots in a greenhouse. These plants were evaluated for reduced spread of FHB infection (type 2 resistance). At flowering, a basal flower in the third spikelet from the tip of the primary spike of each plant (FIG. 4) was inoculated with 10 μL of 50,000 spores/mL of Fusarium graminerarum/H₂O spore suspension. The disease spread, measured by the number of diseased spikelets, was recorded 21 days after inoculation.

The F_{2:3} seedlings were also genotyped with co-segregating markers for Bdv3, Qfhs.pur-7EL, and Fhb1. Selections were made based on the presence of the three genes of interest as determined by molecular markers, and a low score based on the spread of FHB disease (type 2 resistance) following inoculation with F. graminerarum. Ten F_{3:4} seeds from selected F_{2:3} plants were planted in flats. At the two-leaf stage, the F_{3:4} seedlings were exposed to viruliferous aphids, Rhopalosiphum padi, carrying two different strains of luteoviruses, BYDV-RPV and BYDV-PAV. The aphids were allowed to feed on the plants for approximately 48 hours after which they were terminated by spraying with the insecticide Dimethoate. Tissue from the infested plants was harvested 14 days following exposure to the viruliferous aphids. The virus titer was determined using an enzyme-linked immunosorbent assay (ELISA) test following the procedures described by Anderson et al. (1998, p. 852).

### Marker Screening With Labeled Primers (TABLES 1A and B)

Fluorescence-tagged SSR marker primers and capillary electrophoresis were used to genotype the F_{3:4} seedlings for Bdv3, Qfhs.pur-7EL and Fhb1. Primers were labeled with either 6-carboxy-fluorescine (FAM) or tetrachloro-6-carboxy-fluorescine (TET) by Applied Biosystems (Foster City, Calif.) following published procedures with slight modifications (Hansson and Kawabe, 2005; Schuelke, 2000). PCR procedures were followed as known to those of skill in the art.

Fluorescence-tagged SSR marker primers and capillary electrophoresis were used to genotype the F_{3:4} seedlings for Bdv3, Qfhs.pur-7EL and Fhb1. Primers were labeled with either 6-carboxy-fluorescine (FAM) or tetrachloro-6-carboxy-fluorescine (TET) by Applied Biosystems (Foster City, Calif.) following published procedures with slight modifications (Hansson and Kawabe, 2005; Schuelke, 2000) PCR procedures were followed as previously described. The final primer concentration used for these reactions was reduced to 0.2 mM. The 25 μL PCR mixtures were amplified in a MyCycler thermal cycler (BioRad, Hercules, Calif.) with an initial denaturation of 94° C. for 2 minutes, 35 cycles of denaturation at 94° C. for 30 seconds, annealing at 52° C. for 40 seconds, extension at 72° C. for 1 minute, and a final extension at 72° C. for 7 minutes.

Following amplification, 10 μL of the PCR product was transferred to a new 96-well plate. That plate was returned to the thermal cycler for a single denaturation step at 95° C. for 5 minutes and immediately placed on ice to chill. Two microliters of the denatured PCR product were then transferred to another 96-well plate where each well contained 9 μL of a 99% Hi-Di Formamide (Applied Biosystems, Foster City, Calif.) and 1% GeneScan-500 LIZ size standard (Applied Biosystems, Foster City, Calif.) mixture. The plates were then transported to the Purdue University Genotyping center for capillary electrophoresis. The output files were evaluated using GeneMarker® software. The final primer concentration used for these reactions was reduced to 0.2 mM. The 25 μL PCR mixtures were amplified in a MyCycler thermal cycler (BioRad, Hercules, Calif.) with an initial denaturation of 94° C. for 2 minutes, 35 cycles of denaturation at 94° C. for 30 seconds, annealing at 52° C. for 40 seconds, extension at 72° C. for 1 minute, and a final extension at 72° C. for 7 minutes.

Following amplification, 10 μL of the PCR product was transferred to a new 96-well plate. That plate was returned to the thermal cycler for a single denaturation step at 95° C. for 5 minutes and immediately placed on ice to chill. Two microliters of the denatured PCR product were then transferred to another 96-well plate where each well contained 9 μL of a 99% Hi-Di Formamide (Applied Biosystems, Foster City, Calif.) and 1% GeneScan-500 LIZ size standard (Applied Biosystems, Foster City, Calif.) mixture. The plates were then transported to the Purdue University Genotyping center for capillary electrophoresis. The output files were evaluated using GeneMarker® software.

### Selection

Families were selected based on their lack of segregation (i.e. all plants of a F_{3:4} family were positive for the DNA markers associated with the introgressed 7E chromosomal segments containing, respectively, Bdv3 and Qfhs.pur-7EL, and marker umn10, associated with Fhb1. The selected plants are inbred progeny of plants in which a recombination occurred, resulting in the combination of Bdv3 and Qfhs.pur-7EL in coupling on chromosome 7D.

Genotypic data revealed eight F_{3:4} families, each represented by at least nine individuals, were positive for all three traits of interest and had no segregation within the families. Overall, there were 172 individuals that were positive for all markers tested. ELISA data was collected for four groups of individuals based on genotypic data. The four groups were as follows: 1) plants that were positive for all three genes of interest, 2) plants that were positive for both FHB-resistance genes and negative for Bdv3, 3) plants that were positive for both Bdv3 and Qfhs.pur-7EL and were heterozygous for Fhb1, and 4) plants that were positive for both Bdv3 and Qfhs.pur-7EL and were negative for Fhb1. The average of the virus titer scores indicated that all groups with Bdv3 were resistant to YD infection and the group without Bdv3 was susceptible. The presence or absence of Fhb1 had no affect on the effectiveness of Bdv3 (Table 3). Plants from the eight families showing no segregation and positive for all three genes/QTL (Bdv3, Qfhs.pur-7EL and Fhb1) are progenitor plants for a wheat line, Line P07117B1-29-7-9-9-4, in which Bdv3 and Qfhs.pur-7EL are combined in coupling on the long arm of chromosome 7D.

TABLE 1
Labeled primers used in marker screening. Primer name, sequence, and
label attached to the 5′ end of the forward primer used in capillary
electrophoresis to determine the presence or absence of the three
genes of interest: Qfhs.pur-7EL (cfa2240), Bdv3 (BDV), and Fhb1 (umn10).
TABLE 1A

| Primer | Sequence | Label at 5′ |
|---|---|---|
| cfa2240-FAM | 5′-6FAM TGC AGC ATG CAT TTT | FAM |
| | AGC TT-3′ | |
| | (SEQ ID NO: 1) | |
| cfa2240-R | 5′-TGC CGC ACT TAT TTG TTC | none |
| | AC-3′ | |
| | (SEQ ID NO: 2) | |
| BDV-TET | 5′-TET-CTT AAC TTC ATT GTT | TET |
| | GAT CTT A-3′ | |
| | (SEQ ID NO: 3) | |
| BDV-R | 5′-CGA CGA ATT CCC AGC TAA | none |
| | ACT AGA CT-3′ | |
| | (SEQ ID NO: 4) | |
| umn10-FAM | 5′-6FAM CGT GGT TCC ACG TCT | FAM |
| | TCT TA-3′ | |
| | (SEQ ID NO: 5) | |
| umn10-R | 5′-TGA AGT TCA TGC CAC GCA | none |
| | TA-3′ | |
| | (SEQ ID NO: 6) | |

TABLE 1B

| | Forward | reverse |
|---|---|---|
| cfa2240 | TGC AGC ATG CAT TTT AGC TT | TGC CGC ACT TAT TTG TTC AC |
| | (SEQ ID NO: 1) | (SEQ ID NO: 2) |
| BDV3 | CTT AAC TTC ATT GTT GAT CTT A | CGA CGA ATT CCC AGC TAA ACT AGA |
| | (SEQ ID NO: 3) | CT |
| | | (SEQ ID NO: 4) |
| BF145935 | CTT CAC CTC CAA GGA GTT CCA C | GCG TAC CTG ATC ACC ACC TTG AAG |
| | (SEQ ID NO: 7) | G |
| | | (SEQ ID NO: 8) |
| BE445653 | GCG TGG TAT CCC ATA TAC CG | GTT CAG CGT CGA CAA CCT TT |
| | (SEQ ID NO: 9) | (SEQ ID NO: 10) |

TABLE 2
Resistance to Fusarium head blight and DNA marker genotypes
of selected F₃ plants with Bdv3 from P961341.

| Entry¹ | Source² | FHB Score³ | cfa 2240⁴ | BF 145935 | BDV⁵ | gwm 493 | gwm 533 |
|---|---|---|---|---|---|---|---|
| 1262-3 | 15003 + 09Fld5 | 0.5 | +⁶ | + | + | + | + |
| 1262-6 | 15003 + 09Fld5 | 0.5 | . | + | + | + | + |
| 1264-1 | 15005 + 09Fld5 | 1 | + | + | + | + | + |
| 1264-2 | 15005 + 09Fld5 | 0.5 | + | + | + | + | + |
| 1264-4 | 15005 + 09Fld5 | 1 | + | + | + | + | + |
| 1267-6 | 15010 + 09Fld5 | 1 | + | + | + | + | + |
| 1268-1 | 15011 + 09Fld5 | 0.5 | . | + | + | + | + |
| 1268-2 | 15011 + 09Fld5 | 1 | + | . | + | + | + |
| 1268-5 | 15011 + 09Fld5 | 1 | . | + | + | + | + |
| 1268-6 | 15011 + 09Fld5 | 0.5 | + | + | + | + | + |
| 1268-7 | 15011 + 09Fld5 | 1 | + | + | + | + | + |
| 1268-8 | 15011 + 09Fld5 | 1 | + | + | + | + | + |
| 1268-12 | 15011 + 09Fld5 | 0.5 | . | + | + | + | + |
| 1268-13 | 15011 + 09Fld5 | 1 | + | + | + | + | + |
| 1268-14 | 15011 + 09Fld5 | 0.5 | + | + | + | + | + |
| 1269-1 | 15012 + 09Fld5 | 1 | + | + | + | + | . |
| 1269-2 | 15012 + 09Fld5 | 0.5 | + | + | + | + | + |
| 1269-3 | 15012 + 09Fld5 | 1 | + | + | + | + | + |
| 1269-5 | 15012 + 09Fld5 | 1 | + | + | + | + | + |
| 1270-3 | 15013 + 09Fld5 | 0.5 | + | + | + | + | + |
| 1271-5 | 15014 + 09Fld5 | 1 | + | + | + | + | + |
| 1271-6 | 15014 + 09Fld5 | 0.5 | . | + | + | + | + |
| 1273-3 | 15018 + 09Fld5 | 1.5 | . | + | + | + | + |
| 1274-2 | 15019 + 09Fld5 | 0.5 | + | + | + | + | + |
| 1274-5 | 15019 + 09Fld5 | 1 | + | + | + | + | + |
| 1274-6 | 15019 + 09Fld5 | 0.5 | + | + | + | + | + |
| 1274-7 | 15019 + 09Fld5 | 1 | + | − | + | + | + |
| 1274-8 | 15019 + 09Fld5 | 1 | + | + | + | + | + |
| 1276-2 | 15021 + 09Fld5 | 1 | + | + | + | + | + |
| 1277-1 | 15022 + 09Fld5 | 1 | + | + | + | + | + |
| 1278-3 | 15023 + 09Fld5 | 0.5 | + | + | + | + | + |
| 1278-5 | 15023 + 09Fld5 | 0.5 | + | + | + | + | + |
| 1279-1 | 15024 + 09Fld5 | 1 | . | + | + | + | + |
| 1279-3 | 15024 + 09Fld5 | 1 | + | + | + | + | + |
| 1280-2 | 15025 + 09Fld5 | 1 | + | + | + | + | + |
| 1280-4 | 15025 + 09Fld5 | 0.5 | . | + | + | + | + |
| 1281-1 | 15026 + 09Fld5 | 1 | + | + | + | + | + |
| 1281-2 | 15026 + 09Fld5 | 0.5 | + | + | + | + | + |
| 1281-3 | 15026 + 09Fld5 | 1 | + | + | + | + | + |
| 1282-1 | 15027 + 09Fld5 | 1 | + | + | + | + | + |
| 1282-4 | 15027 + 09Fld5 | 1.5 | . | + | + | + | + |
| 1283-1 | 15028 + 09Fld5 | 1 | + | + | + | + | + |
| 1283-2 | 15028 + 09Fld5 | 1 | + | + | + | + | + |
| 1283-3 | 15028 + 09Fld5 | 0.5 | . | + | + | + | + |
| 1284-2 | 15029 + 09Fld5 | 0.5 | + | + | + | + | + |
| 1284-3 | 15029 + 09Fld5 | 1 | . | + | + | + | + |
| 1284-4 | 15029 + 09Fld5 | 1 | + | + | + | + | + |
| 1285-3 | 15030 + 09Fld5 | 1 | + | + | + | + | + |
| 1286-2 | 15031 + 09Fld5 | 1.5 | . | + | + | + | + |
| 1286-5 | 15031 + 09Fld5 | 1 | . | + | + | + | . |
| 1287-1 | 15033 + 09Fld5 | 0.5 | + | + | + | + | + |
| 1287-3 | 15033 + 09Fld5 | 0.5 | + | − | + | + | + |
| 1287-4 | 15033 + 09Fld5 | 1 | + | − | + | + | + |
| 1287-5 | 15033 + 09Fld5 | 0.5 | . | + | + | + | + |
| 1288-1 | 15034 + 09Fld5 | 0.5 | + | − | + | + | + |
| 1288-2 | 15034 + 09Fld5 | 0.5 | + | + | + | + | + |
| 1288-3 | 15034 + 09Fld5 | 0.5 | + | + | + | + | + |
| 1288-4 | 15034 + 09Fld5 | 1 | + | − | + | + | + |
| 1288-5 | 15034 + 09Fld5 | 0.5 | + | − | + | + | . |
| 1288-6 | 15034 + 09Fld5 | 0.5 | . | + | + | + | + |
| 1290-2 | 15036 + 09Fld5 | 1.5 | . | + | + | + | + |
| 1290-3 | 15036 + 09Fld5 | 0.5 | + | + | + | + | + |
| 1290-4 | 15036 + 09Fld5 | 0.5 | + | + | + | + | + |
| 1293-2 | 15039 + 09Fld5 | 0.5 | + | + | + | + | + |
| 1203-3 | 15039 + 09Fld5 | 0.5 | + | + | + | + | + |

¹Entry reflects the number assigned to the plants in the greenhouse.
²Source indicates the plot in the field from which the single plants were dug.
³FHB score was the disease rating assigned following point inoculation with FHB spore solution. Numbers are the number of spikelets that became diseased at 21 days (near physiological maturity) after inoculation of one flower at flowering with 10 microliters of a Fusarium graminearum conidiaspore solution of 50,000 conidiaspores/ml dH₂O. A value of 0.5 indicates that the disease did not spread beyond the inoculated flower.
⁴The primers cfa2240 and BF145935 were used to determine the presence of Qfhs.pur-7EL.
⁵SSR marker BDV was used to determine the presence of Bdv3, and the presence of Fhb1 was determined by flanking markers gwm493 and gwm533.
⁶A (+) represents the presence of the gene, (−) the absence of the gene, and (.) indicates that data was not obtained for that entry/marker.

TABLE 3
Summary of ELISA score and DNA marker genotype of F₄ plants.

| Group¹ | N² | ELISA Score³ | Bdv3 | Qfhs.pur-7EL | Fhb1 |
|---|---|---|---|---|---|
| 1 | 176 | 0.022 | + | + | + |
| 2 | 45 | 0.367 | − | + | + |
| 3 | 62 | 0.052 | + | + | +/− |
| 4 | 53 | 0.069 | + | + | − |

¹Individuals of group 1 were positive for all three genes of interest (Bdv3, Qfhs.pur-7EL, and Fhb1). Group 2 was missing Bdv3. Group 3 was heterozygous (+/−) for Fhb1 and group 4 was negative for Fhb1.
²N denotes the number of individuals within a group.
³The ELISA score is an average of the individual scores within each group.

### PUBLICATIONS CITED

The following documents are incorporated by reference to the extent they relate to or describe materials or methods disclosed herein. Specific locations in publications cited appear in the specification.

Anderson, J. A., R. W. Stack, S. Liu, B. L. Waldron, A. D. Fjeld, C. Coyne, B. Moreno-Sevilla, J. M. Fetch, Q. J. Song, P. B. Cregan, and R. C. Frohberg. 2001. DNA markers for Fusarium head blight resistance QTLs its two wheat populations. Theor and Appl Genet 102:1164-1168.

Anderson, J. M., D. L. Bucholtz, A. E. Greene, M. G. Francki, S. M. Gray, H. Sharma, H. W. Ohm, and K. L. Perry. 1998. Characterization of wheatgrass-derived barley yellow dwarf virus resistance in a wheat alien chromosome substitution line. Phytopathology 88:851-855.

Ayala, L., M. Henry, D. Gonzalez-de-Leon, M. van Ginkel, A. Mujeeb-Kazi, B. Keller, and M. Khairallah. 2001. A diagnostic molecular marker allowing the study of Th. intermedium-derived resistance to BYDV in bread wheat segregating populations. Theoretical and Applied Genetics 102:942-949.

Crasta, O. R., M. G. Francki, D. B. Bucholtz, H. C. Sharma, J. Zhang, R. C. Wang, H. W. Ohm, and J. M. Anderson. 2000. Identification and characterization of wheat-wheatgrass translocation lines and localization of barley yellow dwarf virus resistance. Genome 43:698-706.

Hossain K G, et al., 2004.pp A Chromosome bin map of 2148 EST loci of wheat homeologous group 7. Genetics 168:687-699.

Kong, L., J. M. Anderson, and H. W. Ohm. 2009. Segregation distortion in common wheat of a segment of Thinopyrum intermedium chromosome 7E carrying Bdv3 and development of a Bdv3 marker. Plant Breeding.

Ohm, H. W., J. M. Anderson, H. C. Sharma, L. Ayala, N. Thompson, and J. J. Uphaus. 2005. Registration of yellow dwarf viruses resistant wheat germplasm line P961341. Crop Science 45:805-806.

Roder, M. S., V. Korzun, K. Wendehake, J. Plaschke, M. H. Tixier, P. Leroy, and M. W. Ganal. 1998. A microsatellite map of wheat. Genetics 149:2007-2023.

Sharma, H., H. Ohm, L. Goulart, R. Lister, R. Appels, and O. Benlhabib. 1995. Introgression and characterization of barley yellow dwarf virus resistance from Thinopyrum intermedium into wheat. Genome 38:406-13.

Shen, X., and H. Ohm. 2006. Fusarium head blight resistance derived from Lophopyrum elongatum chromosome 7E and its augmentation with Fhb1 in wheat. Plant Breeding 125:424-429.

Shen, X. R., and H. Ohm. 2007. Molecular mapping of Thinopyrum-derived Fusarium head blight resistance in common wheat. Molecular Breeding 20:131-140.

Song, Q. J., E. W. Fickus, and P. B. Cregan. 2002. Characterization of trinucleotide SSR motifs in wheat. Theoretical and Applied Genetics 104:286-293.

Sourdille P. et al., 2004. Microsatellite-based deletion bin system for the establishment of genetic-physical map relationship in wheat (Triticum aestivum L.). Funct Integr Genomics 4:12-25.

1. A wheat plant comprising the resistance genes Bdv3 and the QTL (Qfhs.pur-7EL).

2. The wheat plant of claim 1 further comprising Fhb1.

3. The wheat plant of claim 1 is inbred.

4. The wheat plant in claim 1 comprises a marker selected from the group consisting of umn 10, BDV3, BE445653, BF145935, cfa2240, and combinations thereof.

5. The wheat plant of claim 1 wherein the resistance genes are present in coupling in chromosome 7D.

6. The wheat plant of claim 1 is substantially resistant to yellow dwarf virus and fusarium head blight fungus.

7. The wheat plant of claim 1 is non-genetically modified or engineered.

8. The wheat plant of claim 2 further comprising one or more additional resistance genes.

9. A wheat seed comprising the resistance genes Bdv3 and the QTL (Qfhs.pur-7EL).

10. The wheat seed of claim 9, wherein wheat germ of the seed does not contain any heterologous chromosomes.

11. A method to improve crops by using the wheat plant of claim 1, whereby crops are resistant to both yellow dwarf virus and fusarium head blight fungus.

12. The wheat plant of claim 1 obtained by crossing the wheat lines designated P961341 and 275-4.

13. The wheat plant of claim 2 is inbred.


SEQUENCE LISTING (10 sequences)

SEQ ID NO: 1 - DNA, 20 bp, Artificial Sequence
  source: /note=“Description of Artificial Sequence Synthetic primer”
tgcagcatgcattttagctt

SEQ ID NO: 2 - DNA, 20 bp, Artificial Sequence
  source: /note=“Description of Artificial Sequence Synthetic primer”
tgccgcacttatttgttcac

SEQ ID NO: 3 - DNA, 22 bp, Artificial Sequence
  source: /note=“Description of Artificial Sequence Synthetic primer”
cttaacttcattgttgatctta

SEQ ID NO: 4 - DNA, 26 bp, Artificial Sequence
  source: /note=“Description of Artificial Sequence Synthetic primer”
cgacgaattcccagctaaactagact

SEQ ID NO: 5 - DNA, 20 bp, Artificial Sequence
  source: /note=“Description of Artificial Sequence Synthetic primer”
cgtggttccacgtcttctta

SEQ ID NO: 6 - DNA, 20 bp, Artificial Sequence
  source: /note=“Description of Artificial Sequence Synthetic primer”
tgaagttcatgccacgcata

SEQ ID NO: 7 - DNA, 22 bp, Artificial Sequence
  source: /note=“Description of Artificial Sequence Synthetic primer”
cttcacctccaaggagttccac

SEQ ID NO: 8 - DNA, 25 bp, Artificial Sequence
  source: /note=“Description of Artificial Sequence Synthetic primer”
gcgtacctgatcaccaccttgaagg

SEQ ID NO: 9 - DNA, 20 bp, Artificial Sequence
  source: /note=“Description of Artificial Sequence Synthetic primer”
gcgtggtatcccatataccg

SEQ ID NO: 10 - DNA, 20 bp, Artificial Sequence
  source: /note=“Description of Artificial Sequence Synthetic primer”
gttcagcgtcgacaaccttt